(19) United States
(12) Patent Application Publication
(10) Pub. No.: US 2015/0059765 A1
(43) Pub. Date: Mar. 5, 2015

(54) IMMOBILIZATION SYSTEM

(71) Applicant: Peter M. Larson, Granville, OH (US)

(72) Inventor: Peter M. Larson, Granville, OH (US)

(21) Appl. No.: 14/194,940

(22) Filed: Mar. 3, 2014

Related U.S. Application Data
(63) Continuation-in-part of application No. 14/010,816, filed on Aug. 27, 2013.

(51) Int. Cl. A61F 5/37

(52) U.S. Cl. CPC A61F 5/3707
USPC 128/845

(57) ABSTRACT

An immobilization system for a body, or body part, is provided, having a fixed form support and one or more shells that generally form an outlining structure of the body part to be immobilized. A formable support, having minimal mass, is molded and interposed between the fixed form support and shells to form a closely fitted support for the body part. The shell or shells may be removed from the system after molding, so that patient comfort is improved and total mass of the system is reduced, without compromise of the degree of immobilization. A retention mask, which also may be molded to conform to the body or body part, may be utilized to further stabilize positioning of the system.

### CROSS-REFERENCE TO RELATED APPLICATIONS

This application is a continuation-in-part of U.S. patent application Ser. No. 14/010,816; filed Aug. 27, 2013; which is incorporated as if totally rewritten herein.

### STATEMENT REGARDING FEDERALLY SPONSORED RESEARCH OR DEVELOPMENT

Not applicable.

### TECHNICAL FIELD

The present disclosure relates generally to an immobilization system, in particular, for making an anatomically high-accuracy moldable immobilization system well-suited for patient immobilization during radiotherapy or other applications requiring a high degree of accuracy in patient positioning, especially for radiotherapy of the head and neck region.

### BACKGROUND OF THE INVENTION

The present invention relates to an immobilization system, for persons whose bodies or body parts are required to be retained in a particular position or attitude. In particular, a need exists for a simple and easy to use immobilization system that allows repetitive patient positioning with a high degree of accurate repeatability across time, for example, wherein radiation treatments must be directed at a certain anatomical point across different times.

The use of low-temperature thermoplastics for patient positioning is well-known and dates back to splinting devices invented in the 1960's (Larson, U.S. Pat. No. 5,540,876). Splints are heated, usually in hot water, to a temperature of about 160° F., whereupon they become pliable and can be molded by hand directly on the patient's body part. These devices are well known in the field of occupational therapy and include splints with padding or cushioning material laminated to the thermoplastic to provide comfort against the patient's skin.

Plastic materials have been successfully used in the past for making splints, casts and the like. U.S. Pat. No. 3,490,444 describes the use of thermoplastic polydienes like transpolyisoprene and transpolychloroprene, which melt between 140° F. (60° Celsius) and 212° F. (100° Celsius), and which harden by crystallization at about 140° F. (60° Celsius), such that these plastics can be formed for use as a body supporting member. Poly (epsilon-caprolactone) (PCL) has also been found to be an excellent splint or cast material (U.S. Pat. No. 4,144,223). Polyurethanes based on prepolymers of poly (epsilon-caprolactone) have also been used (U.S. Pat. No. 4,316,457).

As described in earlier patents, the polymers can be heated in hot water at a temperature usually exceeding 122° F. (50° Celsius) and up to about 212° F. (100° Celsius), whereby they become soft, self-adherent and sufficiently pliable to be deformed and shaped as a cast, splint or protective device. When allowed to cool in air to about 140° F. (40° Celsius), the materials will remain pliable, moldable and cohesive for a period of several minutes, exhibiting a hysteresis, as described in U.S. Pat. No. 3,490,444. During this time the splint, cast or device can be molded directly to the patient without discomfort, and the shaped plastic sets hard by crystallization to assume a rigid form as a useful body support member or protective device.

Splints and casts made of the aforementioned materials provide good support strength, due to the hardness of the cooled materials. PCL, for example cools to a hardness of between 45 and 55 (Shore D), very suitable for a splint or cast, but too hard and uncomfortable for a pillow or a neck brace, for example. Cushioning fabrics are often used with splints to mitigate the hardness of the thermoplastic against the skin.

The aforementioned thermoplastics by themselves are also ill-suited for moldable pillows and formable underlying body supports because of the impracticality of heating and forming times found with suitably thick materials, as can be seen in the Example below. A 3.2 mm splinting sheet of PCL can typically be heated to forming in about a minute in hot water at 160° F. A device of 3 cm thickness or more would take 15 minutes or more to heat, and then a half hour or more to cool to hardness.

In the field of radiation therapy, precise patient positioning is essential for treatment accuracy. An additional requirement is that patients be precisely re-positioned for repeated radiation treatments. Repositioning to an accuracy within millimeters, or even less, is generally desirable. This requires the positioning to be reproduced accurately each time the patient undergoes a treatment. Low-temperature thermoplastic masks in conjunction with other positioning methods are often used for such positioning. Masks are heated to a temperature of about 160° F. (71° C.), and formed directly on to the patients head or other body part. The masks may be affixed to a table supporting the patient and cooled to form a firm mask holding the patient steady for treatment. After treatment, the mask may be removed. When the patient returns for the next treatment, the mask is releasably reattached, holding the patient in a reproduced position for treatment.

Various masks are used for radiation therapy treatments, including stereotactic head masks holding the top and bottom of the patient's head (Vilsmeier, U.S. Pat. No. 5,702,406). Another method of stabilizing the patient's head position includes the use of a moldable cushion or pillow. Hirano (U.S. Pat. No. 6,254,959) teaches a method of making a position-retaining device utilizing a mixture of elastic granules and water-curable resin. The resin and granule mixture is encased in a fabric to make a pillow, cushion or patient support device that can be shaped to a head or other body part and then hardened by adding water to the resin. The cushion is stored in a sealed package before use to prevent premature hardening due to atmospheric or other environmental moisture. When removed from the package and exposed to water the cushion begins to harden. It is placed under the patient's head or other body part to conform to the patient and also to the underlying support structure. The cushion then hardens to become a secure conforming positioning device, suitable for reproducible treatment positioning.

The water-activated resin devices are also known to have spheroidal bodies inside them such as relatively small plastic beads having a diameter of from 1 to 5 mm, which are mixed in with the resin to form a slurry-like material. The slurry is surrounded by a fabric barrier to prevent patient contact with the slurry and to provide patient comfort. Water is applied to the fabric and seeps in to the slurry to activate the hardening of the resin. A significant limitation of such devices is that once hardened, the devices cannot be modified or remolded. Additionally, the devices must be kept scrupulously dry until ready for use. Such water-active resin devices are also not suitable for changes in conformation during a course of time. If positioning needs to be adjusted, the device must be discarded and a new one made.

Another method of patient stabilization is the use of a vacuum apparatus, which is a hermetically sealed bag containing sphere-like bodies, such as relatively small plastic beads having a diameter of from 1 to 5 mm. The patient is placed on the bag, causing the beads to be displaced and conform around the patient. A vacuum pump may be then connected to the bag, and air is evacuated from the bag through a valve that can be closed to prevent air from re-entering the bag. This creates a vacuum state in the interior of the bag, which prevents the sphere-like bodies from moving, thereby holding the bag and the patient in a fixed position. This positioning device is suitable for repeat treatments but not necessarily for accurate repositioning of the patient at another time. This vacuum apparatus is also known to be used to position patients for other medical procedures, such as in operating rooms. A limitation of the device is that the bags can be easily punctured by a scalpel, syringe or knife. Should any air leak into the device, it loses its conformation and positioning must begin anew. The device also has no elasticity or air-permeability and is not comfortable for extended treatment times.

Another goal of patient immobilization systems for radiation therapies is the avoidance of as much mass surrounding the patient's body as possible, as the mass tends to attenuate a radiotherapy beam. Therefore, the avoidance of unnecessary bulk in the immobilization system confers an advantage over more bulky systems. However, at the same time, systems have found it difficult to achieve adequate stabilization without using relatively high-mass immobilization devices.

Recently, a new thermoplastic splint has been described in U.S. patent application Ser. No. 14/010,816; that, in some embodiments, is useful as a component of the immobilization system described herein. That splint may be heat molded, in relatively thin embodiments, to conform to the human anatomy with a high degree of accuracy, and therefore may be useful in achieving highly stable and repeatable patient positioning. Because of its relatively low mass, such a thermoplastic splint also contributes minimal interference with potential radiotherapy applications.

### SUMMARY OF THE INVENTION

The disclosed invention relates to methods to for making an immobilization system. In one embodiment, the system includes a fixation apparatus, which as seen in FIG. XX, may be configured as a generally flat plate that is removable from other equipment, but replaceable in nearly exactly the same position to the same equipment. A primary forming shell is in reversible contact with the fixation apparatus and may generally be shaped in rough outline to surround a body part. In some embodiments a secondary sizing shell is at least partially adjacent to the primary forming shell interior surface, conforming generally to the same body part.

A fixed-form support supports a formable support, so that in general, the fixed-form support generally conforms to a body part, and the formable support closely conforms to the same body part. The formable support may be molded nearly exactly in conformation to a particular patient. This near-exact conformation may supply the close reproducibility across time for individual patients.

The primary forming shell and the secondary sizing shell may be removable from the immobilization system in an operating position. This confers a number of advantages, increasing patient comfort and decreasing the overall mass of the immobilization system, while maintaining accurate patient support. The primary forming shell and secondary sizing shell provide for the forming of the formable support in conjunction with the fixed-form support. The generally “nesting” construction of the primary forming shell and secondary sizing shell allows the formable support to be made from a material having sufficient rigidity to hold the body part firmly, but with minimal mass. When the primary forming shell and secondary sizing shell are removed from the system while in an operating position, the minimized mass of the formable support remains.

Especially for those systems intended for use on the human head and neck area, there may be a retention mask, molded to the face or head contours of the patient, holding the desired body parts firmly to the formable support. This allows for high reproducibility in positioning.

In some embodiments, the at least one secondary sizing shell further includes an exterior secondary sizing shell. This generally “nested” construction of the shells allows a high degree of individualization of shell sizes, as one or more of the secondary sizing shells may be employed, or removed, so as to minimize the mass of the formable support needed to adequately hold a body part. The fixed-form support surface may also be anatomically sculpted to conform to a human body part, which also decreases the necessary mass of the formable support.

### BRIEF DESCRIPTION OF THE ILLUSTRATIONS

Without limiting the scope of the as disclosed herein and referring now to the drawings and figures:

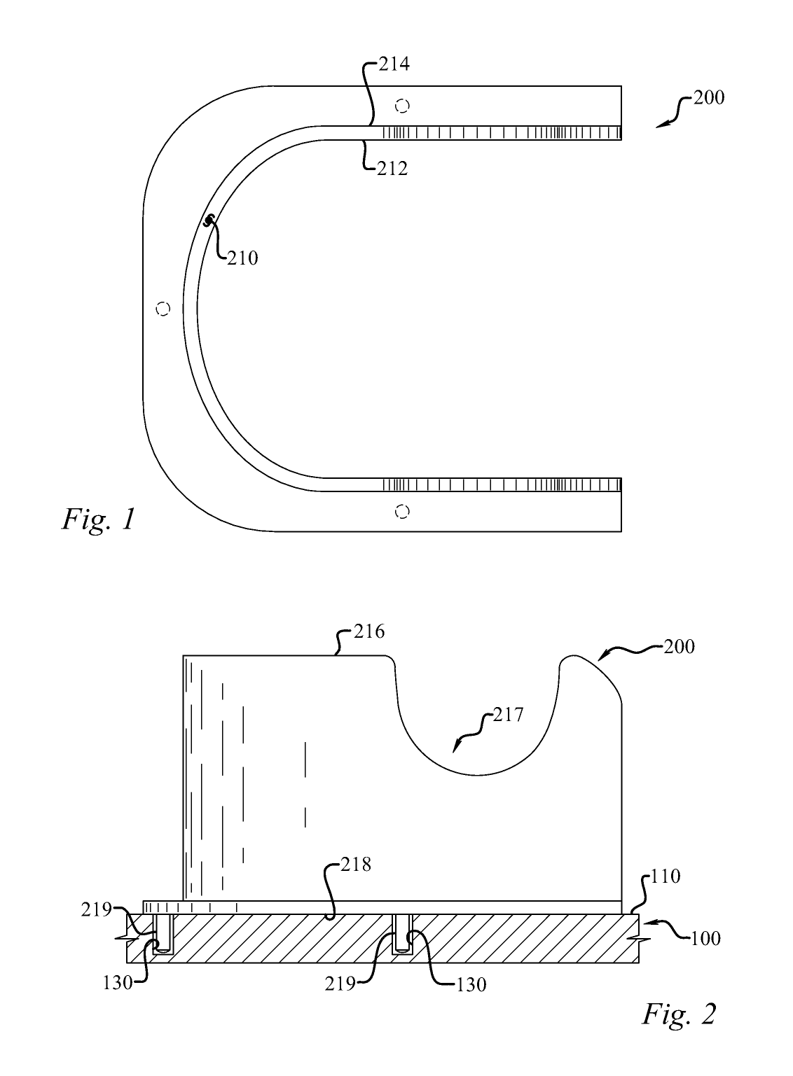

FIG. 1 is a top plan view of an embodiment of one component of an immobilization system;

FIG. 2 is a side elevation view of an embodiment of two components of an immobilization system;

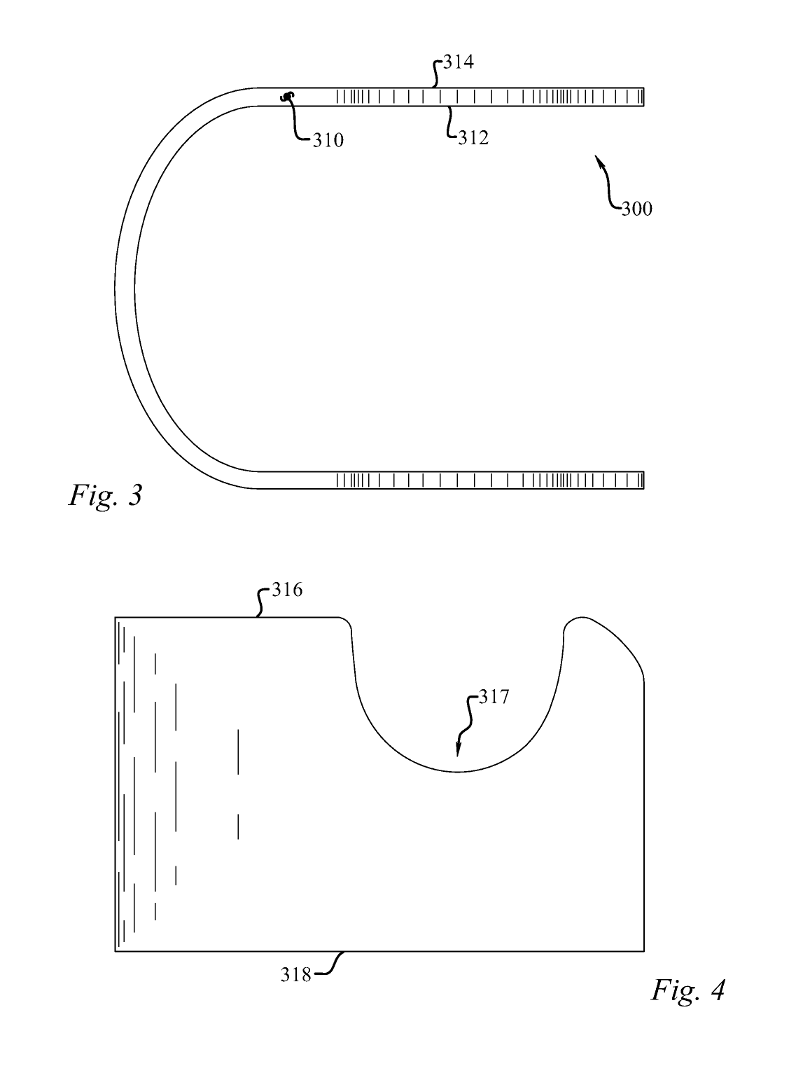

FIG. 3 is a top plan view of an embodiment of another component of an immobilization system;

FIG. 4 is a side elevation view of the embodiment of FIG. 3;

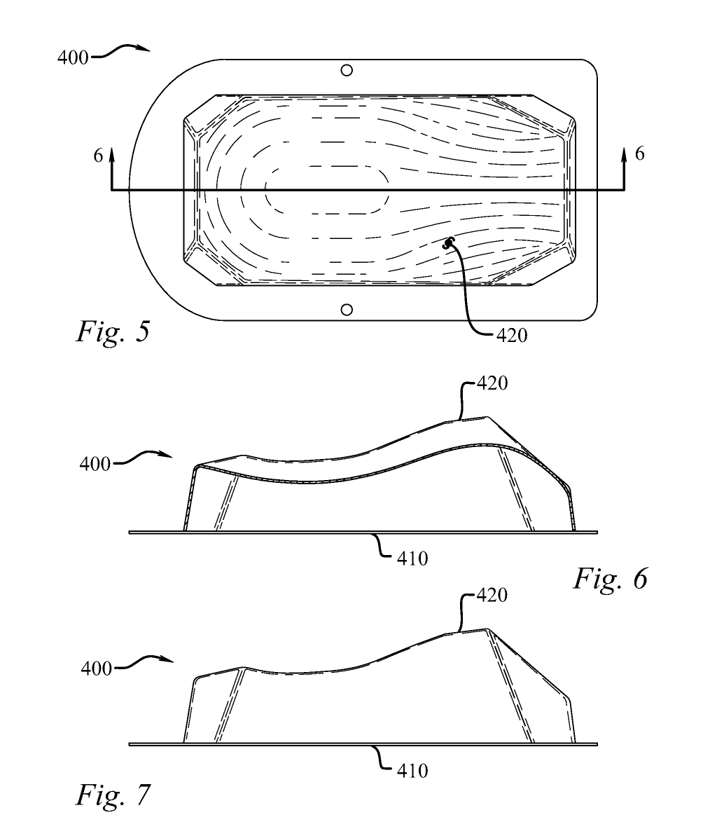

FIG. 5 is a top plan view of an embodiment of another component of an immobilization system;

FIG. 6 is a sectional view taken along line 6-6 of FIG. 5;

FIG. 7 is a side elevation view of the embodiment of FIG. 5;

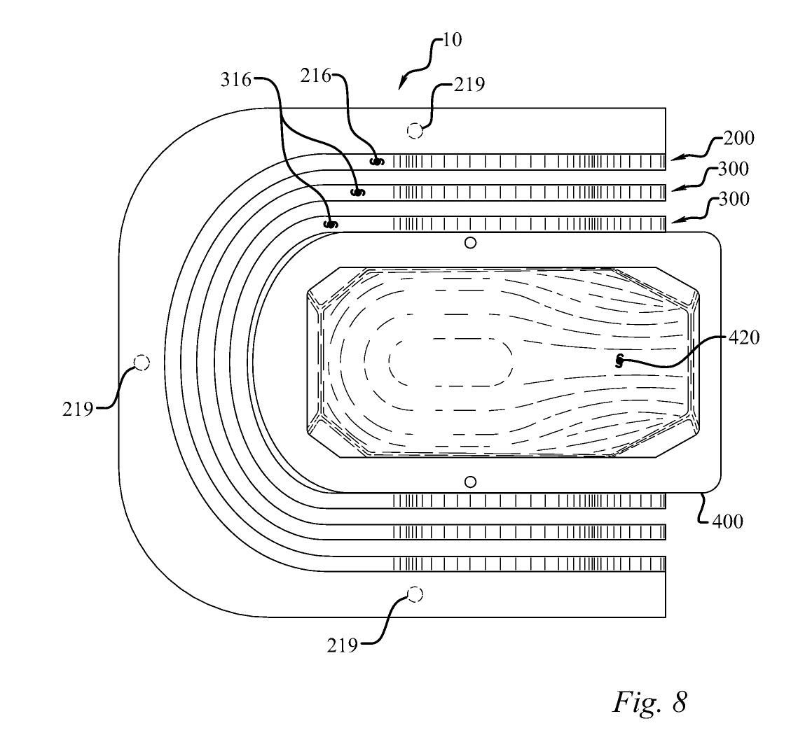

FIG. 8 is a top plan view of a plurality of elements of an immobilization system;

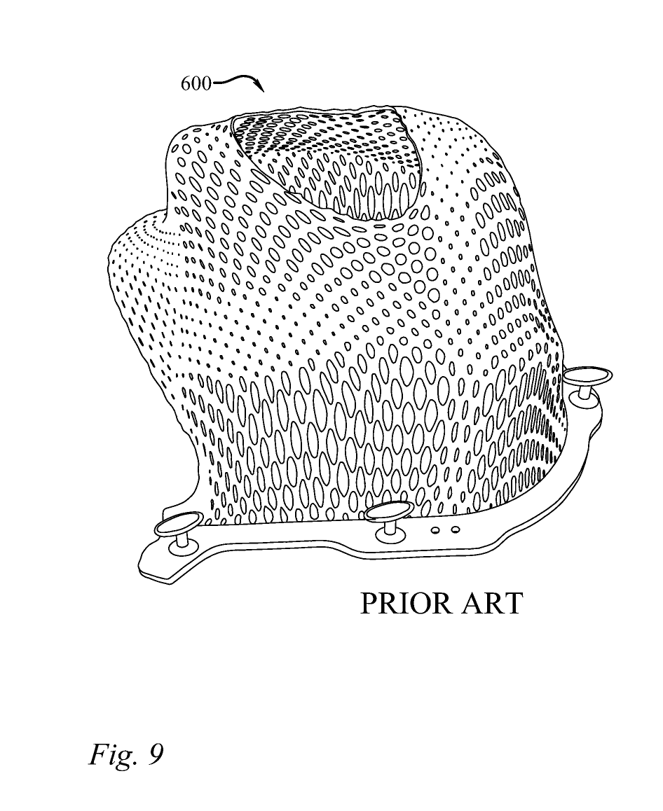

FIG. 9 is a perspective view of another (prior art) component of an immobilization system;

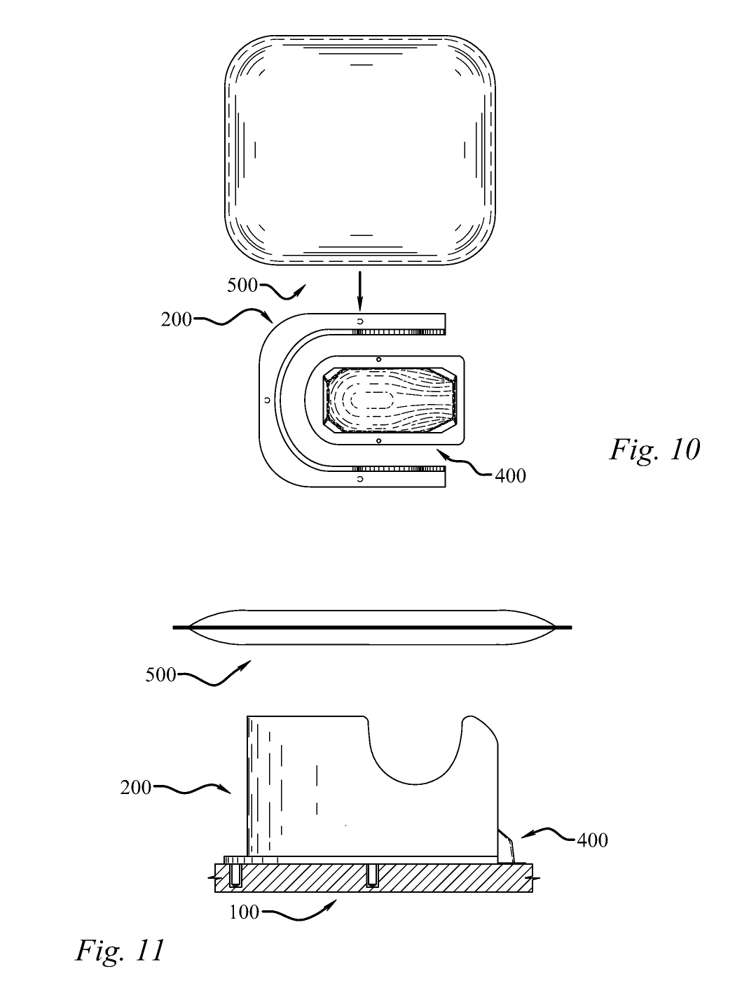

FIG. 10 is a top plan exploded view of three components, partially assembled, of an embodiment of an immobilization system;

FIG. 11 is a side exploded view of four components, partially assembled, of an embodiment of an immobilization system;

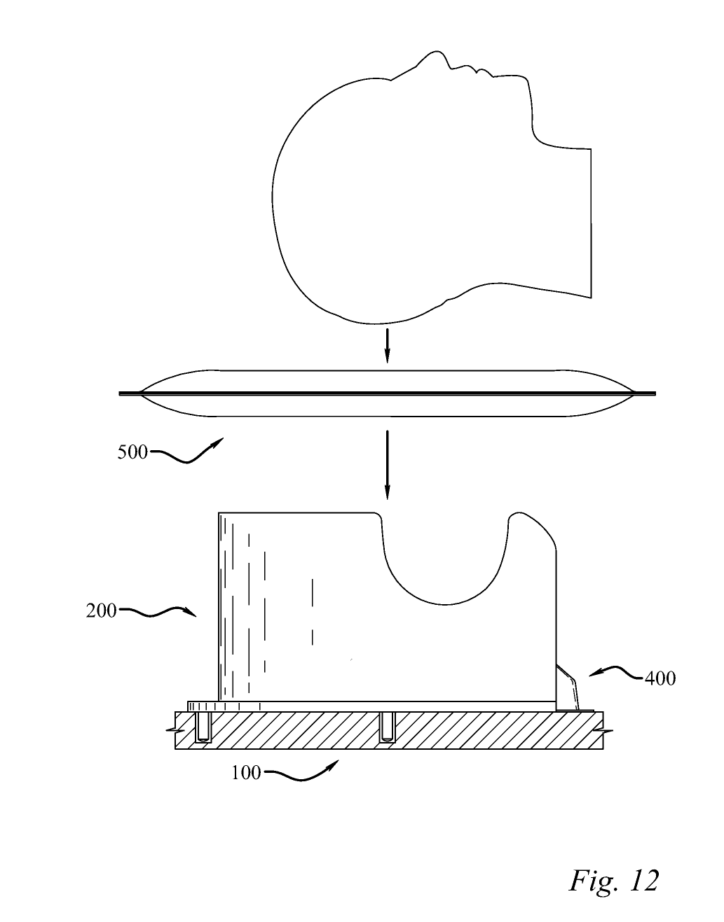

FIG. 12 is a side exploded view of a partially assembled embodiment of an immobilization system, showing a preparatory position prior to the fitting of the system to a human head; and

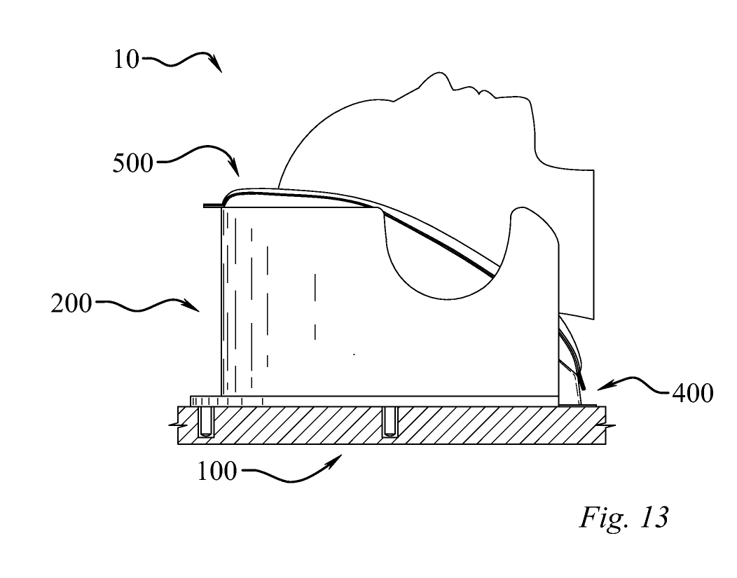

FIG. 13 is a side view of an assembled embodiment of an immobilization system, showing a position subsequent to the fitting of the system to a human head.

These illustrations are provided to assist in the understanding of the exemplary embodiments of the method of forming an immobilization system and materials related thereto described in more detail below and should not be construed as unduly limiting the specification. In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

What is claimed then, as seen in FIGS. 1-13, is an immobilization system (10) for highly reproducible patient positioning. In one embodiment, the system (10) includes a fixation apparatus (100) with at least an upper surface (110), as seen well in FIG. 2. This may be a generally flat plate that is removable from other equipment, but may represent any surface to which the system is generally attached. The fixation apparatus (100) has various apparatus attachment fixtures (130), which may or not be placed on or as a part of the upper surface (100), that are reversibly attachable to a primary forming shell (200), seen well in conjunction with the fixation apparatus (100) in FIG. 1. The primary forming shell may have a primary forming shell sidewall (210), a primary forming shell sidewall interior surface (212), a primary forming shell sidewall exterior surface (214), a primary forming shell sidewall upper edge (216) and a primary forming shell sidewall engagement surface (218). The primary forming shell (200) is in reversible contact with the fixation apparatus (100) at the primary forming shell sidewall engagement surface (218), and may generally be shaped in rough outline to surround a body part.

By way of example and not limitation, as seen in some illustrated embodiments, such as FIG. 1, the primary forming shell (200) has a general horseshoe shape so as to generally conform to the outline of a human head. However, there is no reason that the primary forming shell (200) may not be generally shaped to conform to the outline of any other body part, and these shapes are expressly intended to be included as possible embodiments. In the embodiment as seen in FIGS. 2, 8, 10, 11, 12 and 13, the primary forming shell sidewall engagement surface (218) is seen as a primary forming shell sidewall lower edge (218), however, this is not intended as a requirement in any way. By way of example and not limitation, the primary forming shell engagement surface (218) may be formed on any practicable surface of the primary forming shell (200), including as being a part of some other primary forming shell surface. By way of further example only, in those embodiments where the primary forming shell (200) might be formed in a general bowl or other such shape, the primary forming shell (200) may not form a distinct primary forming shell lower edge (218), but in such an embodiment the primary forming shell engagement surface (218) might then comprise a point or portion of the primary forming shell exterior surface (214), or in some other area of the primary forming shell (200) as would be known to one skilled in the art, including embodiments where the primary forming shell engagement surface (218) may be fully coextensive with the primary forming shell exterior surface (214).

As seen well in FIG. 4, in some embodiments there is at least one secondary sizing shell (300) having a secondary sizing shell sidewall (310), a secondary sizing shell sidewall interior surface (312), a secondary sizing shell exterior surface (314), a secondary sizing shell upper edge (316) and a secondary sizing shell engagement surface (318) wherein the at least one secondary sizing shell exterior surface (314) is at least partially adjacent to the primary forming shell interior surface (212). The term “adjacent” in this specification does not require or imply that the named structures are touching, either in whole or in part. There may well be an air-gap between them, and the possibility of some intervening structure, at least in whole or in part, separating the two is not unexpected. It is simply intended that the primary forming shell (200) and the secondary sizing shell (300) conform generally to the same body part, and that the secondary sizing shell (300) be positioned closer to an ultimate patient or patient body part than the primary forming shell (200).

In some embodiments, seen well in FIGS. 5-8, a fixed-form support (400) may have a fixed-form support base (410) and a fixed-form support surface (420). The fixed form support (400) may be at least partially adjacent to at least one secondary sizing shell interior surface (312). Again, there is no preclusion of an air-gap between the fixed form support (400) and the at least one secondary sizing shell interior surface (312), as seen in FIG. 8, and it is even possible that an intervening structure slightly separate the two in whole or in part. Again, it is simply intended that the two be of the same general shape, to conform generally to the same body part, and that the fixed form support (400) be positioned closer to an ultimate patient or patient body part than the primary forming shell (200).

There may be a formable support (500), seen well in FIGS. 10-13, in reversible contact with the fixed-form support surface (420), so that in general, the fixed-form support (400) generally conforms to a body part, and the formable support (500) closely conforms to the same body part. In fact, in many embodiments, as seen in FIGS. 12-13, the formable support (500) is molded nearly exactly in conformation to a particular patient. This near-exact conformation may supply the close reproducibility across time for individual patients. The formable support (500), may by way of example and not limitation, be formed similarly to the moldable splint described in co-pending U.S. patent application Ser. No. 14/010,816, but this is certainly not required.

The primary forming shell (200) and the at least one secondary sizing shell (300) may be removable from the immobilization system (10) in an operating position. This confers a number of advantages, increasing patient comfort and decreasing the overall mass of the immobilization system, while maintaining accurate patient support. The primary forming shell (200) and secondary sizing shell (300) provide for the forming of the formable support (500) in conjunction with the fixed-form support (400) as seen well in FIGS. 12-13. The generally “nesting” construction of the primary forming shell (200) and secondary sizing shell (300), as seen well in FIG. 8, allows the formable support (500) to be made from a material having sufficient rigidity but minimal mass. In those embodiments where the primary forming shell (200) and secondary sizing shell (300) are removable from the system (10) while in an operating position, the minimized mass of the formable support (500) remains.

In other embodiments, especially but not only those intended for use on the human head and neck area, there may be a retention mask (600), as seen well in FIG. 9, having a plurality of mask attachment fixtures (630). Retention masks (600) may be shaped in predetermined shapes and sized so as to be useful in a wide variety of applications with a wide variety of patients. At least some of the mask attachment fixtures are reversibly attached to the fixation apparatus (100), fixing the mask (600) to the fixation apparatus (100), generally but not necessarily at the fixation support surface (110). The retention mask (600) reversibly covers at least a portion of the fixation apparatus upper surface (110), and thence at least a portion of a patient, when in an operating position. The formable support (500) and the retention mask (600) may include a thermoplastic material for easier forming.

To ease in molding, the primary forming shell sidewall (210) and/or at least one secondary sizing shell sidewall (310) may have at least one anatomical cut-out (217, 317), as seen well in FIGS. 2, 4, 11, 12 and 13. As illustrated herein, the shells (210, 310) are shown with anatomical cut-outs (217, 317) meant roughly to accommodate the human ear. However, it is expressly reserved that such cut-outs (217, 317) be possible in any application in which they improve the moldability of the system (10).

In some embodiments, the at least one secondary sizing shell (300) further includes an exterior secondary sizing shell (300) and an interior secondary sizing shell (300), as seen well in FIG. 8. Such construction facilitates sizing of the system (10) for optimal patient use. For example, a patient with a smaller head may utilize one or more secondary sizing shells (300) in order to size the system (10) most closely to their personal head size. This allows easier sizing by the user molding the system (10), in that multiple-sized primary forming shells (200) need not be stocked, and also allows the system to be molded using a minimally-sized formable support (500), thereby decreasing the overall mass of the system (10). In further embodiments that decrease mass and improve patient comfort, the fixed-form support surface (420) may be anatomically sculpted to conform to a human body part, thereby also minimizes the necessary mass of the formable support (500). The fixed-form support (400) may be formed as a removable piece from the system (10) and therefore may have a fixed-form support base (410) in reversible contact with the fixation apparatus upper surface (110), as seen well in FIGS. 12-13.

In many embodiments, it is intended that the primary forming shell upper edge (216) may be more distant at all points orthogonally measured from the fixation apparatus upper surface (110), than the fixed form support surface (420) orthogonally measured is from the fixation apparatus upper surface (110) at any point. This is partially seen in FIGS. 12 and 13, and this tends to put the fixed form support surface (420) lower, in a relative sense, that the primary forming shell upper edge (216), so that the formable support (500) is finally molded more or less into a bowl shape.

In other embodiment, an immobilization system (10) can include a fixation apparatus (100), having at least an upper surface (110) and a plurality of apparatus attachment fixtures (130) reversibly attachable to a primary forming shell (200), again as seen well in FIG. 2. The primary forming shell (200) may have a primary forming shell sidewall (210), a primary forming shell sidewall interior surface (212), a primary forming shell sidewall exterior surface (214), a primary forming shell sidewall upper edge (216) and a primary forming shell sidewall engagement surface (218) such that the primary forming shell (200) may be placed in reversible contact with the fixation apparatus (100) at the primary forming shell sidewall engagement surface (218) at the primary forming shell sidewall engagement surface (218), as seen well in FIGS. 1 and 2.

These embodiments can have a fixed-form support (400) having a fixed-form support base (410) and a fixed-form support surface (420), again wherein the fixed form support (400) can be at least partially adjacent to the secondary sizing shell interior surface (312). There can be a formable support (500), seen well in FIGS. 5-8, in reversible contact with the fixed-form support surface (420); and the primary forming shell (200) may be removable from the immobilization system (10) in an operating position. Such embodiments may be particularly useful where the body part size is relatively close to the size of the primary forming shell (200). In embodiments where the body part may fit relatively loosely in the primary forming shell (200), at least one secondary sizing shell (300), seen well in FIG. 8, having a secondary sizing shell sidewall (310), a secondary sizing shell sidewall interior surface (312), a secondary sizing shell exterior surface (314), a secondary sizing shell upper edge (316) and a secondary sizing shell engagement surface (318) may be provided. In general, in such embodiments, the at least one secondary sizing shell exterior surface (314) is at least partially adjacent to the primary forming shell interior surface (212).

Again to improve patient comfort and minimize system (10) mass, the fixed-form support surface (420) may be anatomically sculpted to conform to a human body part, again as seen well in FIGS. 5-7, and moldabily is increased when the formable support (500) further includes a thermoplastic material.

Especially in those embodiments intended for head and neck area use, the system (10) can include a retention mask (600), seen in FIG. 9, having a plurality of mask attachment fixtures (630) where at least some of the mask attachment fixtures (630) are reversibly attached to the fixation apparatus, generally at the apparatus surface (100). The retention mask (600) thus reversibly covers at least a portion of the fixation apparatus upper surface (110), and therefore a least a portion of any intended target body part, when in an operating position.

Finally, in yet other embodiments, an immobilization system (10) can include a fixation apparatus (100), having at least an upper surface (110) and a plurality of apparatus attachment fixtures (130) reversibly attachable to a primary forming shell (200), as seen well in FIGS. 1 and 2. The primary forming shell (200) may have a primary forming shell sidewall (210), a primary forming shell sidewall interior surface (212), a primary forming shell sidewall exterior surface (214), a primary forming shell sidewall upper edge (216) and a primary forming shell sidewall engagement surface (218). Again with reference to FIGS. 1 and 2, the primary forming shell (200) may be in reversible contact with the fixation apparatus (100) at the primary forming shell sidewall engagement surface (218). A fixed-form support (400), seen well in FIGS. 5-8, with a fixed-form support base (410) and a fixed-form support surface (420), where the fixed-form support surface (420) is anatomically sculpted to conform to a human body part, may be in reversible contact with the fixation apparatus upper surface (110) and a formable support (500) may be in reversible contact with the fixed-form support surface (420). A retention mask (600), seen in FIG. 9, having a plurality of mask attachment fixtures (630) can be provided such that at least some of the mask attachment fixtures (630) are reversibly attached to the fixation apparatus (100) and the retention mask (600) reversibly covers at least a portion of the fixation apparatus upper surface (110) when in an operating position.

In some embodiments, the primary forming shell (200) may be removable from the immobilization system (10) in an operating position. In yet other embodiments, at least one secondary sizing shell (300) may be found, as seen well in FIG. 8, a secondary sizing shell sidewall (310), a secondary sizing shell sidewall interior surface (312), a secondary sizing shell exterior surface (314), a secondary sizing shell upper edge (316) and a secondary sizing shell engagement surface (318). At least one secondary sizing shell exterior surface (314) may be at least partially adjacent to the primary forming shell interior surface (212). The primary forming shell (200) and the at least one secondary sizing shell (300) may be removable from the immobilization system (10) in an operating position.

Numerous alterations, modifications, and variations of the preferred embodiments disclosed herein will be apparent to those skilled in the art and they are all anticipated and contemplated to be within the spirit and scope of the disclosed specification. For example, although specific embodiments have been described in detail, those with skill in the art will understand that the preceding embodiments and variations can be modified to incorporate various types of substitute and or additional or alternative materials, relative arrangement of elements, order of steps and additional steps, and dimensional configurations. Accordingly, even though only few variations of the method and products are described herein, it is to be understood that the practice of such additional modifications and variations and the equivalents thereof, are within the spirit and scope of the method and products as defined in the following claims. The corresponding structures, materials, acts, and equivalents of all means or step plus function elements in the claims below are intended to include any structure, material, or acts for performing the functions in combination with other claimed elements as specifically claimed.

I claim:

1. An immobilization system (10) comprising;
a fixation apparatus (100), having at least an upper surface (110) and a plurality of apparatus attachment fixtures (130) reversibly attachable to;
a primary forming shell (200), having a primary forming shell sidewall (210), a primary forming shell sidewall interior surface (212), a primary forming shell sidewall exterior surface (214), a primary forming shell sidewall upper edge (216) and a primary forming shell sidewall engagement surface (218), wherein the primary forming shell (200) is in reversible contact with the fixation apparatus (100) at the primary forming shell sidewall engagement surface (218);
at least one secondary sizing shell (300) having a secondary sizing shell sidewall (310), a secondary sizing shell sidewall interior surface (312), a secondary sizing shell exterior surface (314), a secondary sizing shell upper edge (316) and a secondary sizing shell engagement surface (318) wherein the at least one secondary sizing shell exterior surface (314) is at least partially adjacent to the primary forming shell interior surface (212);
a fixed-form support (400) having a fixed-form support base (410) and a fixed-form support surface (420), wherein the fixed form support (400) is at least partially adjacent to the at least one secondary sizing shell interior surface (312);
a formable support (500) in reversible contact with the fixed-form support surface (420); and
wherein the primary forming shell (200) and the at least one secondary sizing shell (300) are removable from the immobilization system (10) in an operating position.

2. The system of claim 1, further comprising a retention mask (600) having a plurality of mask attachment fixtures (630), wherein at least some of the mask attachment fixtures (630) are reversibly attached to the fixation apparatus (100) and the retention mask (600) reversibly covers at least a portion of the fixation apparatus upper surface (110) when in an operating position.

3. The system of claim 1, wherein the primary forming shell sidewall (210) has at least one anatomical cut-out (217).

4. The system of claim 1, wherein the at least one secondary sizing shell sidewall (310) has at least one anatomical cut-out (317).

5. The system of claim 1, wherein the at least one secondary sizing shell (300) further comprises an exterior secondary sizing shell (300) and an interior secondary sizing shell (300).

6. The system of claim 1, wherein the fixed-form support surface (420) is anatomically sculpted to conform to a human body part.

7. The system of claim 1, wherein the fixed form support (400) further comprises a fixed-form support base (410) in reversible contact with the fixation apparatus upper surface (110).

8. The system according to claim 1, wherein the formable support (500) further comprises a thermoplastic material.

9. The system according to claim 2, wherein the retention mask (600) further comprises a thermoplastic material.

10. The system according to claim 1, wherein the primary forming shell upper edge (216) is more distant at all points orthogonally measured from the fixation apparatus upper surface (110) than the fixed form support surface (420) orthogonally measured is from the fixation apparatus upper surface (110) at any point.

11. An immobilization system (10) comprising;
a fixation apparatus (100), having at least an upper surface (110) and a plurality of apparatus attachment fixtures (130) reversibly attachable to;
a primary forming shell (200), having a primary forming shell sidewall (210), a primary forming shell sidewall interior surface (212), a primary forming shell sidewall exterior surface (214), a primary forming shell sidewall upper edge (216) and a primary forming shell sidewall engagement surface (218) in reversible contact with the fixation apparatus (100) at the primary forming shell sidewall engagement surface (218);
a fixed-form support (400) having a fixed-form support base (410) and a fixed-form support surface (420), wherein the fixed form support (400) is at least partially adjacent to the secondary sizing shell interior surface (312);
a formable support (500) in reversible contact with the fixed-form support surface (420); and
wherein the primary forming shell (200) is removable from the immobilization system (10) in an operating position.

12. The system according to claim 11, further comprising at least one secondary sizing shell (300) having a secondary sizing shell sidewall (310), a secondary sizing shell sidewall interior surface (312), a secondary sizing shell exterior surface (314), a secondary sizing shell upper edge (316) and a secondary sizing shell engagement surface (318) wherein at least one secondary sizing shell exterior surface (314) is at least partially adjacent to the primary forming shell interior surface (212).

13. The system according to claim 11, wherein the fixed-form support surface (420) is anatomically sculpted to conform to a human body part.

14. The system according to claim 11, wherein the formable support (500) further comprises a thermoplastic material.

15. The system according to claim 11, further comprising a retention mask (600) having a plurality of mask attachment fixtures (630), wherein at least some of the mask attachment fixtures are reversibly attached to the fixation apparatus (100) and the retention mask (600) reversibly covers at least a portion of the fixation apparatus upper surface (110) when in an operating position.

16. An immobilization system (10) comprising;
a fixation apparatus (100), having at least an upper surface (110) and a plurality of apparatus attachment fixtures (130) reversibly attachable to;
a primary forming shell (200), having a primary forming shell sidewall (210), a primary forming shell sidewall interior surface (212), a primary forming shell sidewall exterior surface (214), a primary forming shell sidewall upper edge (216) and a primary forming shell sidewall engagement surface (218) in reversible contact with the fixation apparatus (100) at the primary forming shell sidewall engagement surface (218);
a fixed-form support (400) having a fixed-form support base (410) and a fixed-form support surface (420), wherein the fixed form support (400) is in reversible contact with the fixation apparatus upper surface (110);
a formable support (500) in reversible contact with the fixed-form support surface (420); and
a retention mask (600) having a plurality of mask attachment fixtures (630), wherein at least some of the mask attachment fixtures are reversibly attached to the fixation apparatus (100) and the retention mask (600) reversibly covers at least a portion of the fixation apparatus upper surface (110) when in an operating position.

17. The system according to claim 16 wherein the primary forming shell (200) is removable from the immobilization system (10) in an operating position.

18. The system according to claim 16, further comprising at least one secondary sizing shell (300) having a secondary sizing shell sidewall (310), a secondary sizing shell sidewall interior surface (312), a secondary sizing shell exterior surface (314), a secondary sizing shell upper edge (316) and a secondary sizing shell engagement surface (318) wherein at least one secondary sizing shell exterior surface (314) is at least partially adjacent to the primary forming shell interior surface (212).

19. The system according to claim 16, wherein the fixed-form support surface (420) is anatomically sculpted to conform to a human body part.

20. The system according to claim 18, wherein the primary forming shell (200) and the at least one secondary sizing shell (300) are removable from the immobilization system (10) in an operating position.
